(19) United States
(12) Patent Application Publication
(10) Pub. No.: US 2025/0381401 A1
(43) Pub. Date: Dec. 18, 2025

(54) METHOD AND DEVICE FOR INDUCING THE PERCEPTION OF COLOR IN A VISUAL PROSTHESIS

(71) Applicant: IRIDIUM MEDICAL TECHNOLOGY CO., LTD., Hsinchu (TW)

(72) Inventors: FENG-HSIUNG HSU, Hsinchu (TW); LONG-SHENG FAN, Hsinchu (TW); SAN-YUAN LIN, Hsinchu (TW); LEE LIN, Hsinchu (TW); YUNG-CHAN CHEN, Hsinchu (TW)

(21) Appl. No.: 19/314,289

(22) Filed: Aug. 29, 2025

Related U.S. Application Data
(63) Continuation-in-part of application No. 17/704,815, filed on Mar. 25, 2022, now Pat. No. 12,415,065.
Application No. 17/704,815 is a continuation-in-part of application No. 16/230,218, filed on Dec. 21, 2018, now abandoned.
(60) Provisional application No. 62/610,004, filed on Dec. 22, 2017.

(51) Int. Cl. A61N 1/36; A61F 2/14; A61F 9/08; A61N 1/05

(52) U.S. Cl. CPC A61N 1/36057; A61F 2/14; A61F 9/08; A61N 1/0543; A61N 1/36046; A61N 1/3615; A61N 1/36171; A61N 1/36175; A61N 1/36178

(57) ABSTRACT

An artificial retinal prosthesis is disclosed for electrically stimulating a retina of an eye by spatiotemporal electrical stimulation to provide color visual perception. The prosthesis comprises a plurality of pixel group units, each including a main pixel unit and at least one auxiliary pixel unit. The main and auxiliary pixel units are driven according to different sequences of cycles, each cycle comprising a dark period followed by a lighted period, such that the pixel units intermittently output lighted pulses. During the lighted periods, the main pixel unit is nominally turned on for at least one lighted duration except for an off duration while the auxiliary pixel units are continuously turned on for the entire lighted period. During the dark periods, both the main pixel unit and the auxiliary pixel units are continuously turned off for the entire dark period.

### CROSS-REFERENCE TO RELATED APPLICATION

The present application is a continuation-in-part application of U.S. patent application Ser. No. 17/704,815, filed on Mar. 25, 2022, which is a continuation-in-part application of U.S. patent application Ser. No. 16/230,218, filed on Dec. 21, 2018, which claims the benefit of U.S. Provisional Patent Application No. 62/610,004, entitled “System for Artificial Retina Prosthesis,” which was filed on Dec. 22, 2017, and the disclosure of which is incorporated herein by reference in its entirety.

### FIELD OF THE INVENTION

The present invention relates to an artificial retinal prosthesis, and more particularly to an artificial retinal prosthesis for providing color visual perception.

### BACKGROUND OF THE INVENTION

Currently, among the patients with visual deterioration, some patients choose to implant an artificial retina to improve their vision. At present, expensive artificial retinas of the commercial standard with low pixels have a limited improvement on the quality of life of patients. In view of this, many companies as well as academic and research institutes have begun to actively invest in the improvement of microsystem for artificial retina.

In order to give the users a more comfortable visual experience, many R&D teams are actively making improvements on the image resolution. For example, U.S. Pat. No. 7,751,896 B2, U.S. Pat. No. 6,804,560 B2 improve the signal transmission in the artificial retina by adding components such as an amplifier or a photosensitive reference component to the circuit, so that the electrical stimulation signals of the artificial retina are more even, when the patient wears the above artificial retina, it is just like the response of eyes to ambient light conditions under natural conditions. There are also other teams that focus on the colors of the image, hoping to upgrade the conventional artificial retinas that only show black and white images to color images. For example, in U.S. Pat. No. 7,840,274 B2, the artificial retina comprises a color image receiver for receiving a color image and converting the color image into an electrical signal, and an image processing unit coupled to the color image receiver for processing the electrical signal. In the patent, a plurality of pixel electrodes are driven by data from the image processing unit to stimulate the optic nerve by time mode to produce a perception of color images. As far as we know, however, there is no published evidence that the time mode stimulation scheme as described in said patent works universally, reliably, or at all.

At present, a number of pixel units of the artificial retina continues to increase, which has been greatly advanced for artificial retinas having only a few tens of pixel units in the past. In contrast, related researches on artificial retinal systems that provide color visual perception are still at a very early stage, and even though many manufacturers and teams have proposed various artificial retinal systems that provide color visual perception, there is no corresponding product/system that has been manufactured, or it is not good enough to achieve color visual perception after practical operations. Obviously, there is still a lot of room for development in developing artificial retina systems providing color visual perception, depending on the continuous investment and improvement of relevant teams.

### SUMMARY OF THE INVENTION

The invention provides an artificial retinal prosthesis to electrically stimulate a retina of an eye by a spatiotemporal electrical stimulation. The artificial retinal prosthesis comprises a plurality of pixel group units arranged in an array, each of the pixel group units comprising a main pixel unit and at least one auxiliary pixel unit adjacent to the main pixel unit, each of the pixel units having one or more pixels. The main pixel unit and its auxiliary pixel unit are configured to receive signals that represent a visual image.

The main pixel unit and its auxiliary pixel unit are configured to operate according to a first sequence of cycles and a second sequence of cycles respectively, each cycle comprising a dark period followed by a lighted period, such that the pixel units intermittently output lighted pulses during the lighted period, each of the dark period being of comparable duration as the lighted period.

The lighted periods of the main pixel unit and the lighted periods of its auxiliary pixel unit are synchronized, and the dark periods of the main pixel unit and the dark periods of its auxiliary pixel unit are synchronized.

During the lighted periods, the main pixel unit is nominally turned on for at least one lighted duration except for an off duration while the auxiliary pixel units are continuously turned on for the entire lighted period, and wherein during the dark periods, both the main pixel unit and the auxiliary pixel units are continuously turned off for the entire dark period.

The artificial retinal prosthesis is configured to provide at least one of the following color percepts:

reddish color when the main pixel units are configured such that the off duration of the lighted periods followed by the lighted duration for the remaining of the lighted periods of its main pixel units;

greenish color when the main pixel units are configured such that the initial lighted duration is followed by the off duration, which is in turn followed by the subsequent lighted duration for the remaining portion of the lighted periods; or

bluish color perceived when the main pixel units are configured such that the lighted duration of the lighted periods followed by the off duration for the remaining of the lighted periods of its main pixel units.

At least one complete cycle is required to induce the color percepts.

The invention further provides an artificial retinal prosthesis to electrically stimulate a retina of an eye by a spatiotemporal electrical stimulation, comprising a plurality of pixel group units arranged in an array, each of the pixel group units comprising a main pixel unit and at least one auxiliary pixel unit adjacent to the main pixel unit, each of the pixel units having one or more pixels. The main pixel unit and its auxiliary pixel unit are configured to receive signals that represent a visual image.

The main pixel unit and its auxiliary pixel unit are configured to operate according to a first sequence of cycles and a second sequence of cycles respectively, each cycle comprising a lighted period followed by a dark period, such that the pixel units intermittently output lighted pulses during the lighted period, each of the dark period being of comparable duration as the lighted period.

The lighted periods of the main pixel unit and the lighted periods of its auxiliary pixel unit are synchronized, and the dark periods of the main pixel unit and the dark periods of its auxiliary pixel unit are synchronized.

During the lighted periods, the main pixel unit and the auxiliary pixel units are continuously turned on for the entire lighted period, and during the dark periods, the main pixel unit is nominally turned off for at least one off duration except for a lighted duration while the auxiliary pixel units is continuously turned off for the entire dark period.

The artificial retinal prosthesis is configured to provide at least one of the following color percepts:

first color when the main pixel units are configured such that the off duration of the lighted periods followed by the lighted duration for the remaining of the lighted periods of its main pixel units;

second color when the main pixel units are configured such that the initial lighted duration is followed by the off duration, which is in turn followed by the subsequent lighted duration for the remaining portion of the lighted periods; or

third color when the main pixel units are configured such that the lighted duration of the lighted periods followed by the off duration for the remaining of the lighted periods of its main pixel units;

At least one complete cycle is required to induce the color percepts.

The artificial retinal prosthesis of the present invention causes the stimulation of the pixel electrodes and the spectrum of the external visual image entering the user's eyes to change synchronously along with different time sequences and different spatiotemporal distributions of each retinal cell, thereby stimulating the patient's retinal cells to provide the patient with a color image perception that assists the patient in truly obtaining RGB color vision. The spatiotemporal stimulation creates color perception in essential the same way as the so-called Fechner Color effect.

### BRIEF DESCRIPTION OF THE DRAWINGS

FIG. 1 is a schematic diagram of the operation of a system for artificial retinal prosthesis with color vision according to an embodiment of the present invention;

FIG. 2 is a color shutter structure according to an embodiment of the present invention;

FIG. 3 is a schematic view showing an electric field applied to the color shutter structure of FIG. 2 of the present invention;

FIG. 4 is a schematic diagram of activation of row-to-row of the system for artificial retinal prosthesis with color vision according to an embodiment of the present invention;

FIG. 5 is a schematic view showing the states in which pixel electrodes are turned on and off according to embodiment 3 of the present invention;

FIG. 6 is a schematic view showing the states in which the pixel electrodes are turned on and off according to embodiment 4 of the present invention;

FIG. 7 illustrates an artificial retinal prosthesis according to an exemplary embodiment of the invention;

FIG. 8 illustrates a pixel group unit with stimulation cycle according to an exemplary embodiment of the invention;

FIG. 9 illustrates various stimulation cycle for the main pixel unit and the surrounding pixel unit according to an exemplary embodiment of the invention;

FIGS. 10A-10C illustrate various stimulation cycles for the main pixel unit and its auxiliary pixel unit according to an exemplary embodiment of the invention;

FIGS. 11A-11C illustrate various stimulation cycles for the main pixel unit and its auxiliary pixel unit according to an exemplary embodiment of the invention;

FIG. 12 illustrates the spacing between the pixel units according to an exemplary embodiment of the invention; and

FIG. 13 illustrates a configuration of the pixel group units according to an exemplary embodiment of the invention.

### DETAILED DESCRIPTION OF THE PREFERRED EMBODIMENTS

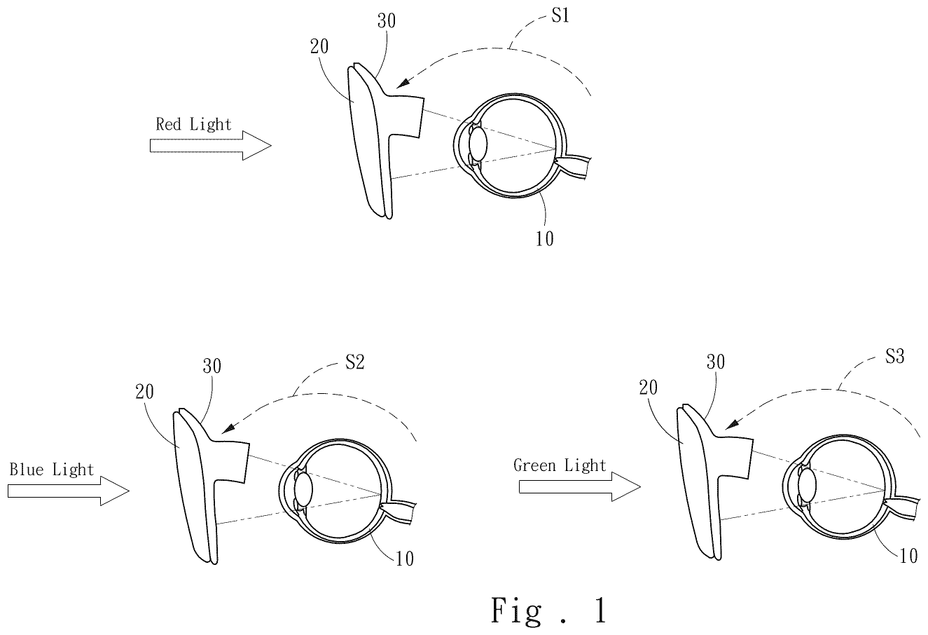

Referring to FIG. 1. A system for artificial retinal prosthesis with color vision in an embodiment of the present invention mainly comprises an artificial retinal prosthesis 10 and a color shutter 20, and the color shutter 20 is fitted on a goggle 30. In other embodiments, the color shutter 20 can also be fitted to a pair of glasses or other devices that can be worn by a user.

The artificial retinal prosthesis 10 can send a wireless signal to the goggle 30 to control the color shutter 20 of the goggle 30. For example, when the artificial retinal prosthesis 10 needs a red light stimulus, the artificial retinal prosthesis 10 sends a wireless signal S1 to the goggle 30 to activate a red color shutter in the color shutter 20, so that only red light can pass through the red color shutter of the goggle 30 to reach the artificial retinal prosthesis 10. If a blue light stimulus is required, the artificial retinal prosthesis 10 sends a wireless signal S2 to the goggle 30 to activate a blue color shutter in the color shutter 20, so that blue light can pass through the goggle 30 to reach the artificial retinal prosthesis 10. Likewise, when a green light stimulus is required, the artificial retinal prosthesis 10 sends a wireless signal S3 to the goggle 30 to activate a green color shutter, so that green light can pass through the goggle 30 to reach the artificial retinal prosthesis 10. Subsequently, after the artificial retinal prosthesis 10 receives a specific incident light such as red light, blue light, or green light through the color shutter 20, a pixel electrode array in the artificial retinal prosthesis 10 is electrically stimulated by a spatiotemporal electrical stimulation.

When the pixel electrodes in the artificial retinal prosthesis 10 are defined as Pxy according to the spatial positions, such as P11, P12, P13, P22, P23, the above-mentioned “spatiotemporal electrical stimulation” refers to different stimulations given to corresponding optic nerves by different Pxy at different times, for example, P11 and P12 stimulations are given at time point t1, but the remaining pixel electrodes are not.

The artificial retinal prosthesis 10 is disposed on the retina of the eye structure, and can be disposed on the sub-retina or the epi-retina as needed in actual use without particular limitation. This embodiment is disposed on the sub-retina. The artificial retinal prosthesis 10 comprises a plurality of pixel arrays and a processing module disposed correspondingly to the plurality of pixel arrays. Each of the plurality of pixel arrays comprises a substrate and a plurality of sub-pixels disposed on the substrate for receiving a color image. In this embodiment, the substrate can be a thin flexible silicon substrate that can be deformed and bent as desired, so that it can be bent as much as possible into a structure conforming to the shape of a human eye and disposed in the eye of a patient.

In actual manufacturing, for example, the substrate can be fabricated based on a manufacturing process using a Silicon On Insulator (SOI) chip, and formed by thinning the chip after a Metal-Oxide-semiconductor (MOS) fabrication. The processing module can include a correlated double sampling unit (CDS), an analog-to-digital converter (ADC), a digital core, and a digital-to-analog converter (DAC) to process a signal of the pixel array. However, the components included in the processing module are not limited to the above components, technicians of this field can add or delete based on actual needs and designs.

Each of the plurality of sub-pixels comprises at least one pixel electrode, a photodiode, and a circuit architecture electrically connected to the photodiode. After an incident light emit to the photodiode, the incident light is converted into an electric charge and a photovoltaic potential, and a light-induced electrical stimulation signal is generated according to an intensity ratio of the incident light. The light-induced electrical stimulation signal generates the spatiotemporal electrical stimulation to stimulate the patient's retinal cells, thereby producing a color image.

It should be additionally explained that, in another embodiment of the present invention, the color shutter 20 may not be assembled on the goggle 30, but can be integrated into a single structure with the artificial retinal prosthesis 10. That is, the color shutter 20 can be formed on the pixel array of the artificial retinal prosthesis 10 and can include a plurality of optical shutter units corresponding to different colors. For example, the color shutter 20 can include red shutters formed in a first row to a third row of the pixel array, green shutters formed in a fourth row to a sixth row, and blue shutters formed in a seventh row to a ninth row.

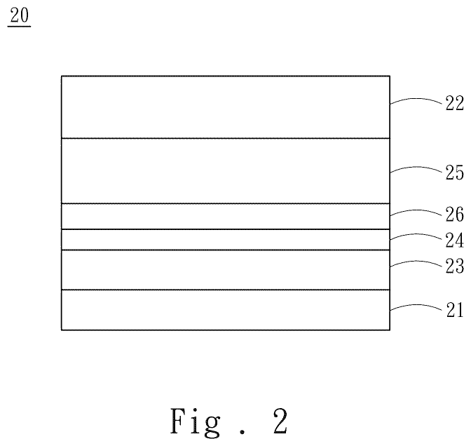

For one of the examples of the color shutter 20, please refer to FIG. 2. The color shutter 20 can include a first substrate 21, a second substrate 22 disposed oppositely to the first substrate 21, an electrode 23 disposed between the first substrate 21 and the second substrate 22, a hydrophobic layer 24 disposed between the electrode 23 and the second substrate 22, a first fluid layer 25 disposed between the hydrophobic layer 24 and the second substrate 22, and a second fluid layer 26 disposed between the hydrophobic layer 24 and the first fluid layer 25, wherein the first fluid layer 25 and the second fluid layer 26 are immiscible with each other.

In this embodiment, the first substrate 21 and the second substrate 22 are transparent and can be formed with the same or different materials, such as glass, resin, polycarbonate (PC), and the like.

The first fluid layer 25 can be a conductive or polarized water or salt solution; and the second fluid layer 26 can be an oily medium, so that when the first fluid layer 25 and the second fluid layer 26 coexist between the second substrate 22 and the hydrophobic layer 24, a two-layer structure can be formed without being miscible. In this embodiment, the second fluid layer 26 can be a mixture of oils with different colors, such as can be selected from a green oil, a red oil, a blue oil, or any combinations of the above oils.

The hydrophobic layer 24 can be a functional layer with low surface energy and high stability, and specifically, can be made of a polymer or a silicon dioxide layer. For example, the polymer used for the hydrophobic layer 24 may be a fluoropolymer such as Cytop or amorphous Teflon, or a hydrocarbon polymer may also be used. If silicon dioxide is used, its surface needs to be treated hydrophobically.

The electrode 23 is disposed on the first substrate 21 to apply a voltage to the first fluid layer 25. The electrode 23 used in the present embodiment is preferably a transparent electrode made of any suitable conductive material such as indium tin oxide (ITO). The above is merely illustrative, and the present invention is not limited thereto, and the color shutter 20 may employ other devices such as a light filter.

In another embodiment of the present invention, the color shutter 20 can further include an optical sensor for sensing ambient light and/or a variable light filter for automatically controlling light passing through the color shutter 20 according to environmental conditions.

The principle used by the color shutter 20 is an electrowetting effect, that is, a wettability of the oily medium on the substrate is controlled by changing a voltage between the oily medium and the hydrophobic layer 24 (insulating layer). More specifically, the oily medium is deformed and displaced by changing a contact angle. The term “wetting” used above refers to the process of a fluid on a solid surface being replaced by another fluid. The fluid on the solid surface (i.e., the hydrophobic layer 24) can diffuse, at this time, the adhesion of the fluid on the solid surface is greater than the cohesion, referred to as “wetting.” Conversely, when the fluid on the solid surface (i.e., the hydrophobic insulating layer) cannot diffuse, the contact surface has a tendency to shrink into a spherical shape, which is called “non-wetting”, and “non-wetting” refers to the adhesion of the fluid on the solid surface being smaller than the cohesion.

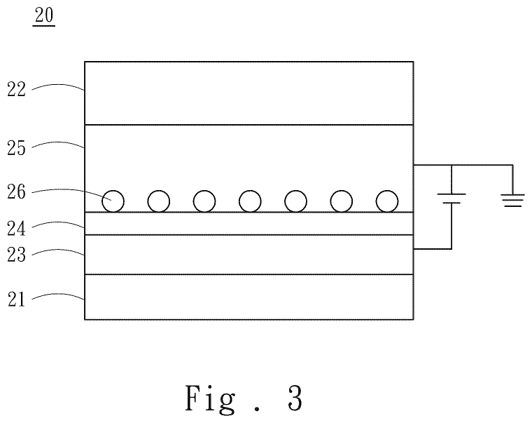

Returning to the present invention, the first fluid layer 25 and the second fluid layer 26 are immiscible with each other without applying an electric field to the fluids (closed state) to form a two-layer structure in which the first fluid layer 25 is diffused to form as a fluid layer adjacent to the second substrate 22; and the second fluid layer 26 also diffuses to form a fluid layer adjacent to the hydrophobic layer 24 and serves as color pixels. However, when an electric field is applied to the fluids (on state), the second fluid layer 26 is broken into small droplets to cause the color shutter 20 to exhibit a transparent color, as shown in FIG. 3.

Therefore, in order to obtain various display results, the second fluid layer 26 (i.e., the oily medium) can be designed to have a desired color, and a surface of the oily fluid can be controlled to change the pixels by controlling the voltage.

In the other embodiment, the anisotropic color pigment particles (say pigment needles) in fluid suspensions could be utilized in an alternative color shutter. Three shutters in tandem, with Yellow, Cyan, and Magenta color pigments, would be needed. Each color shutter would be turned on by applying sufficient large voltage across the fluid to align the particle with the field. Alternatively, another type of color shutter with electrophoretic cells in shutter mode could be used. This is somewhat harder to reach adequate speed, but can work with optimized cells.

When the system for artificial retinal prosthesis with color vision of this embodiment is in use, the color image is converted into the light-induced electrical stimulation signal by the photodiode of the sub-pixel, and the spatiotemporal electrical stimulation is generated to provide the patient with color perception. As a specific example, the spatiotemporal electrical stimulation of about 4 Hz to 8 Hz (preferably 7 Hz) can be divided into seven equally spaced phases within one cycle, producing color sensations of red (R), green (G) and blue (B).

Further explain how to provide the patient with color perception by the spatiotemporal electrical stimulation as below.

### Embodiment 1

In this embodiment, the pixel electrodes in each of the pixel arrays are arranged in 1 column of 9 rows and classified into three groups corresponding to the specific color perceptions. A time series takes 7 equally spaced frames as a cycle, and the cycles per second (cps) can be between 7 and 8, so the frames per second (fps) are between 49 and 56, and the cycle between two of the frames is approximately 20 ms.

Please refer to Table 1, wherein “—” means the pixel electrode is turned off and “|” means the pixel electrode is turned on.

TABLE 1

| | Frame | 1 | 2 | 3 | 4 | 5 | 6 | 7 |
|---|---|---|---|---|---|---|---|---|
| R | Row 1 | — | — | — | \| | \| | \| | \| |
| | Row 2 | — | — | — | — | — | \| | \| |
| | Row 3 | — | — | — | \| | \| | \| | \| |
| G | Row 4 | — | — | — | \| | \| | \| | \| |
| | Row 5 | — | — | — | — | \| | \| | — |
| | Row 6 | — | — | — | \| | \| | \| | \| |
| B | Row 7 | — | — | — | \| | \| | \| | \| |
| | Row 8 | — | — | — | \| | \| | — | — |
| | Row 9 | — | — | — | \| | \| | \| | \| |

For the 3 rows of R/G/B strips, the span is 240 □ m (30 m*8) strip width.

Row 1 to row 9 start rolling at the same time. It can be found from Table 1 that all the pixel electrodes are turned off in frame 1; the pixel electrodes of row 1, row 3 to row 9 are turned on while the other pixel electrodes are turned off in frame 5; while in frame 7, all the pixel electrodes are turned on except for the pixel electrodes of row 5 and row 8 being turned off. Based on the arrangement and operation of the pixel electrodes described above, the patient can perceive colors on the corresponding pixel electrodes, for example, in the cycles from frame 6 to frame 7, the patient can perceive red in the pixel electrodes of row 2.

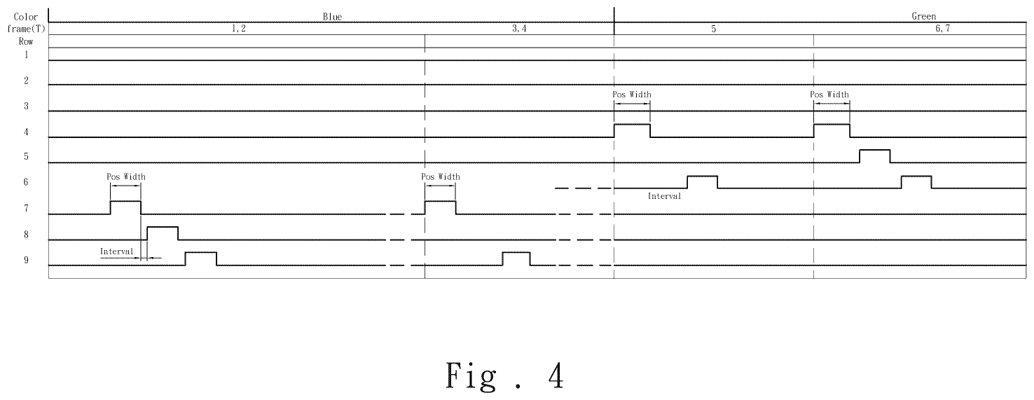

If power attenuation problem is taken into consideration, in other embodiments, electrical stimulations of the above-mentioned “rows” are not simultaneously sent out in the same frame. If all the “rows” in the same frame are enabled at exactly the same time, the artificial retinal prosthesis 10 will consume a very large amount of power and cause a drop in power, even making the artificial retinal prosthesis 10 unable to function properly. In order to avoid the above problem, in the cycles of the same frame, when the state of the pixel electrodes is “|” representing being turned-on, they will be activated row-to-row. That is to say, electrical stimulations of the subsequent rows will slightly lag behind the previous pixel electrode; however, when the state of the pixel electrodes is “—” representing being turned-off, as in the first column to the third column (frame 1 to frame 3) of Table 1 above, the pixel electrodes in the columns cannot be activated and electrical stimulations are not sent out from the columns. Please refer to FIG. 4, where the horizontal axis (x-axis) is time and the vertical axis (y-axis) is electrical signal strength, that is, voltage. “Pulse width” in FIG. 4 refers to pulse duration, and “Interval” represents time delay. In Tables 1 and 2, the time for turning on each of the rows is simultaneous, but in reality, there may be a time difference between turning on each of the rows (such as the time delay of ns level), for example, after row 7 is turned on and off, then it is the turn for row 8 to be turned on and off.

### Embodiment 2

Please refer to Table 2. In this embodiment, the pixel electrodes are arranged in 1 column of 6 rows, and the electrodes are classified into three groups with each of the groups respectively corresponding to a specific color. The setting of the time series in this embodiment is the same as that of Embodiment 1.

TABLE 2

| | Frame | 1 | 2 | 3 | 4 | 5 | 6 | 7 |
|---|---|---|---|---|---|---|---|---|
| R | Row 1 | — | — | — | — | — | \| | \| |
| | Row 2 | — | — | — | \| | \| | \| | \| |
| G | Row 3 | — | — | — | — | \| | \| | — |
| | Row 4 | — | — | — | \| | \| | \| | \| |
| B | Row 5 | — | — | — | \| | \| | — | — |
| | Row 6 | — | — | — | \| | \| | \| | \| |

This embodiment is similar to that of Table 1, the pixel electrodes of row 1 to row 6 are all turned off in frame 1; the pixel electrodes of row 2 to row 6 are all turned on in frame 5, only the pixel electrodes of row 1 are turned off; while in frame 7, all the pixel electrodes are turned on except for the pixel electrodes of row 3 and row 5 being turned off.

### Embodiment 3

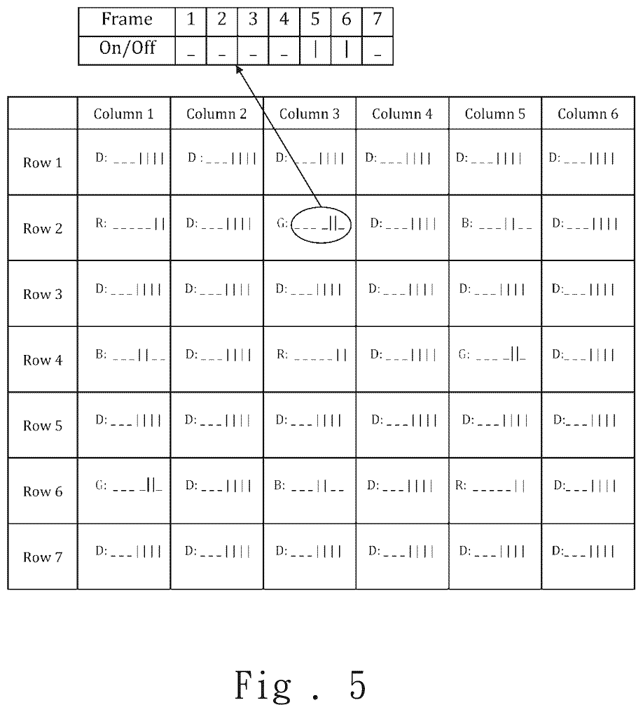

Please refer to FIG. 5, wherein “D” represents a dummy electrode, “R” represents an electrode group that can perceive red correspondingly, “G” represents an electrode group that can perceive green correspondingly, and “B” represents an electrode group that can perceive blue correspondingly. In this embodiment, the pixel electrodes are arranged in 7 rows of 6 columns, and each of the R, G, B pixel electrodes is surrounded by 9 dummy electrodes.

If row 2 of column 3 is taken as an example, all the pixel electrodes are turned off in frame 1 to frame 4, and the surrounding dummy electrodes are also turned off; while the pixel electrodes are turned on in frame 5 to frame 6, so only green light can pass through the goggle 30 to reach the artificial retinal prosthesis 10, and at the same time, the surrounding dummy electrodes are also turned on. According to the operation of the pixel electrodes described above, the patient can have a green visual perception in the pixel electrodes of column 3 and row 2, and by the above arrangement of the dummy electrodes, a visual contrast can be generated between the pixel electrodes corresponding to the specific colors and the surrounding areas thereof, and the effect of enhancing the color perception of the patient is achieved.

### Embodiment 4

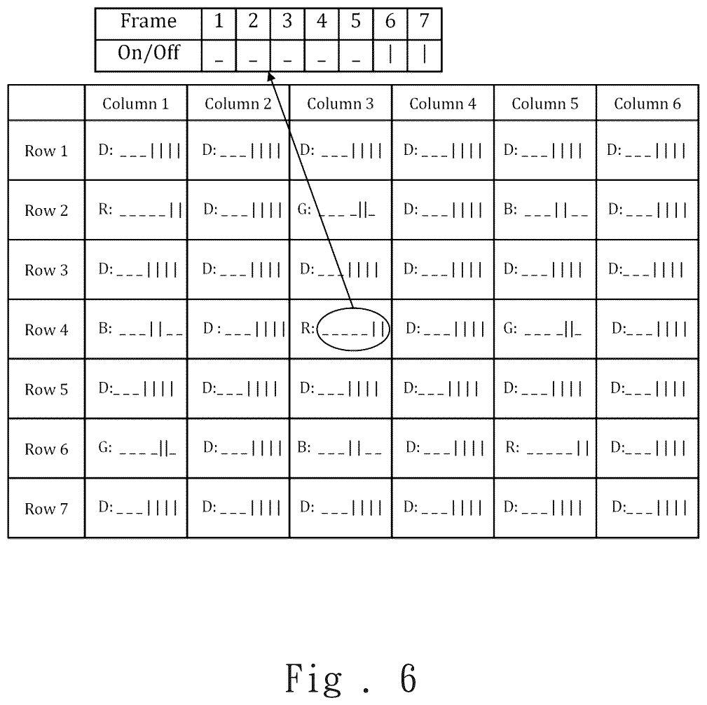

Referring to FIG. 6, “D” represents a dummy electrode, “R” represents an electrode group that can perceive red correspondingly, “G” represents an electrode group that can perceive green correspondingly, and “B” represents an electrode group that can perceive blue correspondingly. In this embodiment, the pixel electrodes are arranged in 7 rows of 6 columns, and each of the R, G, B pixel electrodes is surrounded by 9 dummy electrodes.

The operation of this embodiment is basically the same as that of Embodiment 3. If row 4 of column 3 is taken as an example, all the pixel electrodes are turned off in frame 1 to frame 5, and the surrounding dummy electrodes are also turned off; while the pixel electrodes are turned on in frame 6 to frame 7, so only red light can pass through the goggle 30 to reach the artificial retinal prosthesis 10, and at the same time, the surrounding dummy electrodes are also turned on.

### Embodiment 5

The electrodes of the present embodiment are arranged in 1 column of 132 rows, and are divided into four groups, which respectively are “D” representing a dummy electrode, “R” representing an electrode group that can perceive red correspondingly, “G” representing an electrode group that can perceive green correspondingly, and “B” representing an electrode group that can perceive blue correspondingly. Wherein, each of the R, G, and B pixel electrodes is surrounded by 2 dummy electrodes. A time series takes 12 frames as a cycle, the cycles per second (cps) are 6, so the frames per second (fps) are 72.

In Table 3, “—” means the pixel electrode is turned off and “|” means the pixel electrode is turned on. For example, if it is desired to enable a blue color shutter during frame 1 to frame 4 so that only blue light can pass through the goggle 30 and generate a specific electrical stimulus, in the cycles of frame 1, blue light is used to activate the pixel electrodes of row 7 to row 9, row 16 to row 18, row 25 to row 27, and row 34 to row 36. The operation of this embodiment is also substantially the same as or similar to that of the foregoing embodiment, and thus will not be described herein again.

TABLE 3

| | | Frame | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| | | Color shutter | | | | | | | | | | | |
| | | B color shutter | | | | G color shutter | | | | R color shutter | | | |
| D | Row 1 | — | — | — | — | — | — | — | — | \| | \| | \| | \| |
| R | Row 2 | — | — | — | — | — | — | — | — | — | — | \| | \| |
| D | Row 3 | — | — | — | — | — | — | — | — | \| | \| | \| | \| |
| D | Row 4 | — | — | — | — | \| | \| | \| | \| | — | — | — | — |
| G | Row 5 | — | — | — | — | — | \| | \| | — | — | — | — | — |
| D | Row 6 | — | — | — | — | \| | \| | \| | \| | — | — | — | — |
| D | Row 7 | \| | \| | \| | \| | — | — | — | — | — | — | — | — |
| B | Row 8 | \| | \| | — | — | — | — | — | — | — | — | — | — |
| D | Row 9 | \| | \| | \| | \| | — | — | — | — | — | — | — | — |
| D | Row 10 | — | — | — | — | — | — | — | — | \| | \| | \| | \| |
| R | Row 11 | — | — | — | — | — | — | — | — | — | — | \| | \| |
| D | Row 12 | — | — | — | — | — | — | — | — | \| | \| | \| | \| |
| D | Row 13 | — | — | — | — | \| | \| | \| | \| | — | — | — | — |
| G | Row 14 | — | — | — | — | — | \| | \| | — | — | — | — | — |
| D | Row 15 | — | — | — | — | \| | \| | \| | \| | — | — | — | — |
| D | Row 16 | \| | \| | \| | \| | — | — | — | — | — | — | — | — |
| B | Row 17 | \| | \| | — | — | — | — | — | — | — | — | — | — |
| D | Row 18 | \| | \| | \| | \| | — | — | — | — | — | — | — | — |
| D | Row 19 | — | — | — | — | — | — | — | — | \| | \| | \| | \| |
| R | Row 20 | — | — | — | — | — | — | — | — | — | — | \| | \| |
| D | Row 21 | — | — | — | — | — | — | — | — | \| | \| | \| | \| |
| D | Row 22 | — | — | — | — | \| | \| | \| | \| | — | — | — | — |
| G | Row 23 | — | — | — | — | — | \| | \| | — | — | — | — | — |
| D | Row 24 | — | — | — | — | \| | \| | \| | \| | — | — | — | — |
| D | Row 25 | \| | \| | \| | \| | — | — | — | — | — | — | — | — |
| B | Row 26 | \| | \| | — | — | — | — | — | — | — | — | — | — |
| D | Row 27 | \| | \| | \| | \| | — | — | — | — | — | — | — | — |
| D | Row 28 | — | — | — | — | — | — | — | — | \| | \| | \| | \| |
| R | Row 29 | — | — | — | — | — | — | — | — | — | — | \| | \| |
| D | Row 30 | — | — | — | — | — | — | — | — | \| | \| | \| | \| |
| D | Row 31 | — | — | — | — | \| | \| | \| | \| | — | — | — | — |
| G | Row 32 | — | — | — | — | — | \| | \| | — | — | — | — | — |
| D | Row 33 | — | — | — | — | \| | \| | \| | \| | — | — | — | — |
| D | Row 34 | \| | \| | \| | \| | — | — | — | — | — | — | — | — |
| B | Row 35 | \| | \| | — | — | — | — | — | — | — | — | — | — |
| D | Row 36 | \| | \| | \| | \| | — | — | — | — | — | — | — | — |
| D | Row 37 | — | — | — | — | — | — | — | — | \| | \| | \| | \| |
| R | Row 38 | — | — | — | — | — | — | — | — | — | — | \| | \| |
| D | Row 39 | — | — | — | — | — | — | — | — | \| | \| | \| | \| |
| D | Row 40 | — | — | — | — | \| | \| | \| | \| | — | — | — | — |
| G | Row 41 | — | — | — | — | — | \| | \| | — | — | — | — | — |
| D | Row 42 | — | — | — | — | \| | \| | \| | \| | — | — | — | — |
| D | Row 43 | \| | \| | \| | \| | — | — | — | — | — | — | — | — |
| B | Row 44 | \| | \| | — | — | — | — | — | — | — | — | — | — |
| D | Row 45 | \| | \| | \| | \| | — | — | — | — | — | — | — | — |
| D | Row 46 | — | — | — | — | — | — | — | — | \| | \| | \| | \| |
| R | Row 47 | — | — | — | — | — | — | — | — | — | — | \| | \| |
| D | Row 48 | — | — | — | — | — | — | — | — | \| | \| | \| | \| |
| D | Row 49 | — | — | — | — | \| | \| | \| | \| | — | — | — | — |
| G | Row 50 | — | — | — | — | — | \| | \| | — | — | — | — | — |
| D | Row 51 | — | — | — | — | \| | \| | \| | \| | — | — | — | — |
| D | Row 52 | \| | \| | \| | \| | — | — | — | — | — | — | — | — |
| B | Row 53 | \| | \| | — | — | — | — | — | — | — | — | — | — |
| D | Row 54 | \| | \| | \| | \| | — | — | — | — | — | — | — | — |
| D | Row 55 | — | — | — | — | — | — | — | — | \| | \| | \| | \| |
| R | Row 56 | — | — | — | — | — | — | — | — | — | — | \| | \| |
| D | Row 57 | — | — | — | — | — | — | — | — | \| | \| | \| | \| |
| D | Row 58 | — | — | — | — | \| | \| | \| | \| | — | — | — | — |
| G | Row 59 | — | — | — | — | — | \| | \| | — | — | — | — | — |
| D | Row 60 | — | — | — | — | \| | \| | \| | \| | — | — | — | — |
| D | Row 61 | \| | \| | \| | \| | — | — | — | — | — | — | — | — |
| B | Row 62 | \| | \| | — | — | — | — | — | — | — | — | — | — |
| D | Row 63 | \| | \| | \| | \| | — | — | — | — | — | — | — | — |
| D | Row 64 | — | — | — | — | — | — | — | — | \| | \| | \| | \| |
| R | Row 65 | — | — | — | — | — | — | — | — | — | — | \| | \| |
| D | Row 66 | — | — | — | — | — | — | — | — | \| | \| | \| | \| |
| D | Row 67 | — | — | — | — | \| | \| | \| | \| | — | — | — | — |
| G | Row 68 | — | — | — | — | — | \| | \| | — | — | — | — | — |
| D | Row 69 | — | — | — | — | \| | \| | \| | \| | — | — | — | — |
| D | Row 70 | \| | \| | \| | \| | — | — | — | — | — | — | — | — |
| B | Row 71 | \| | \| | — | — | — | — | — | — | — | — | — | — |
| D | Row 72 | \| | \| | \| | \| | — | — | — | — | — | — | — | — |
| D | Row 73 | — | — | — | — | — | — | — | — | \| | \| | \| | \| |
| R | Row 74 | — | — | — | — | — | — | — | — | — | — | \| | \| |
| D | Row 75 | — | — | — | — | — | — | — | — | \| | \| | \| | \| |
| D | Row 76 | — | — | — | — | \| | \| | \| | \| | — | — | — | — |
| G | Row 77 | — | — | — | — | — | \| | \| | — | — | — | — | — |
| D | Row 78 | — | — | — | — | \| | \| | \| | \| | — | — | — | — |
| D | Row 79 | \| | \| | \| | \| | — | — | — | — | — | — | — | — |
| B | Row 80 | \| | \| | — | — | — | — | — | — | — | — | — | — |
| D | Row 81 | \| | \| | \| | \| | — | — | — | — | — | — | — | — |
| D | Row 82 | — | — | — | — | — | — | — | — | \| | \| | \| | \| |
| R | Row 83 | — | — | — | — | — | — | — | — | — | — | \| | \| |
| D | Row 84 | — | — | — | — | — | — | — | — | \| | \| | \| | \| |
| D | Row 85 | — | — | — | — | \| | \| | \| | \| | — | — | — | — |
| G | Row 86 | — | — | — | — | — | \| | \| | — | — | — | — | — |
| D | Row 87 | — | — | — | — | \| | \| | \| | \| | — | — | — | — |
| D | Row 88 | \| | \| | \| | \| | — | — | — | — | — | — | — | — |
| B | Row 89 | \| | \| | — | — | — | — | — | — | — | — | — | — |
| D | Row 90 | \| | \| | \| | \| | — | — | — | — | — | — | — | — |
| D | Row 91 | — | — | — | — | — | — | — | — | \| | \| | \| | \| |
| R | Row 92 | — | — | — | — | — | — | — | — | — | — | \| | \| |
| D | Row 93 | — | — | — | — | — | — | — | — | \| | \| | \| | \| |
| D | Row 94 | — | — | — | — | \| | \| | \| | \| | — | — | — | — |
| G | Row 95 | — | — | — | — | — | \| | \| | — | — | — | — | — |
| D | Row 96 | — | — | — | — | \| | \| | \| | \| | — | — | — | — |
| D | Row 97 | \| | \| | \| | \| | — | — | — | — | — | — | — | — |
| B | Row 98 | \| | \| | — | — | — | — | — | — | — | — | — | — |
| D | Row 99 | \| | \| | \| | \| | — | — | — | — | — | — | — | — |
| D | Row 100 | — | — | — | — | — | — | — | — | \| | \| | \| | \| |
| R | Row 101 | — | — | — | — | — | — | — | — | — | — | \| | \| |
| D | Row 102 | — | — | — | — | — | — | — | — | \| | \| | \| | \| |
| D | Row 103 | — | — | — | — | \| | \| | \| | \| | — | — | — | — |
| G | Row 104 | — | — | — | — | — | \| | \| | — | — | — | — | — |
| D | Row 105 | — | — | — | — | \| | \| | \| | \| | — | — | — | — |
| D | Row 106 | \| | \| | \| | \| | — | — | — | — | — | — | — | — |
| B | Row 107 | \| | \| | — | — | — | — | — | — | — | — | — | — |
| D | Row 108 | \| | \| | \| | \| | — | — | — | — | — | — | — | — |
| D | Row 109 | — | — | — | — | — | — | — | — | \| | \| | \| | \| |
| R | Row 110 | — | — | — | — | — | — | — | — | — | — | \| | \| |
| D | Row 111 | — | — | — | — | — | — | — | — | \| | \| | \| | \| |
| D | Row 112 | — | — | — | — | \| | \| | \| | \| | — | — | — | — |
| G | Row 113 | — | — | — | — | — | \| | \| | — | — | — | — | — |
| D | Row 114 | — | — | — | — | \| | \| | \| | \| | — | — | — | — |
| D | Row 115 | \| | \| | \| | \| | — | — | — | — | — | — | — | — |
| B | Row 116 | \| | \| | — | — | — | — | — | — | — | — | — | — |
| D | Row 117 | \| | \| | \| | \| | — | — | — | — | — | — | — | — |
| D | Row 118 | — | — | — | — | — | — | — | — | \| | \| | \| | \| |
| R | Row 119 | — | — | — | — | — | — | — | — | — | — | \| | \| |
| D | Row 120 | — | — | — | — | — | — | — | — | \| | \| | \| | \| |
| D | Row 121 | — | — | — | — | \| | \| | \| | \| | — | — | — | — |
| G | Row 122 | — | — | — | — | — | \| | \| | — | — | — | — | — |
| D | Row 123 | — | — | — | — | \| | \| | \| | \| | — | — | — | — |
| D | Row 124 | \| | \| | \| | \| | — | — | — | — | — | — | — | — |
| B | Row 125 | \| | \| | — | — | — | — | — | — | — | — | — | — |
| D | Row 126 | \| | \| | \| | \| | — | — | — | — | — | — | — | — |
| D | Row 127 | — | — | — | — | — | — | — | — | \| | \| | \| | \| |
| R | Row 128 | — | — | — | — | — | — | — | — | — | — | \| | \| |
| D | Row 129 | — | — | — | — | — | — | — | — | \| | \| | \| | \| |
| D | Row 130 | — | — | — | — | \| | \| | \| | \| | — | — | — | — |
| G | Row 131 | — | — | — | — | — | \| | \| | — | — | — | — | — |
| D | Row 132 | — | — | — | — | \| | \| | \| | \| | — | — | — | — |

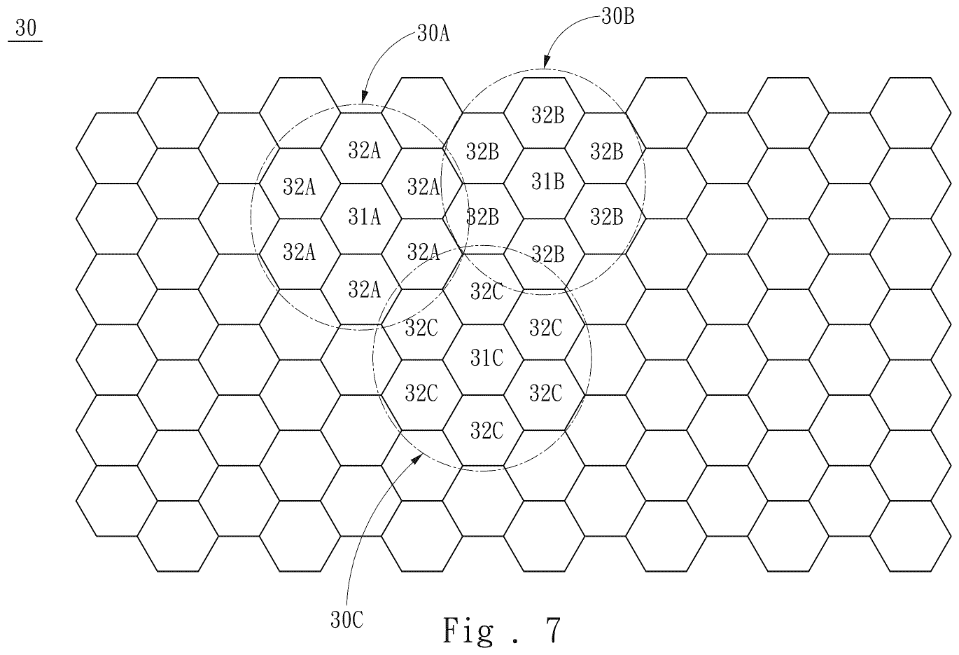

FIG. 7 illustrates an artificial retinal prosthesis according to an exemplary embodiment of the invention. The artificial retinal prosthesis is configurated to electrically stimulate a retina of an eye by a spatiotemporal electrical stimulation. The details of the spatiotemporal electrical stimulation will be described below.

The artificial retinal prosthesis includes a plurality of pixel group units 30A, 30B, 30C arranged in an array. The pixel group units 30A, 30B, 30C are configured to receive an external visual image entering eyes of the user and for inducing perception of different colors.

The pixel group unit 30A comprises a main pixel unit 31A and at least one surrounding pixel unit 32A adjacent to the main pixel unit 30A. The pixel group unit 30B comprises a main pixel unit 31B and at least one surrounding pixel unit 32B adjacent to the main pixel unit 30B. The pixel group unit 30C comprises a main pixel unit 31C and at least one surrounding pixel unit 32C adjacent to the main pixel unit 30C. In this embodiment, the pixel unit is formed in a hexagonal shape. In other embodiments, the pixel unit may be formed in a substantially elongated, circular, elliptic, triangular, quadrangular, pentagonal, heptagonal or octagonal shape.

In this embodiment, the first closest pixel unit to the main pixel is defined as the surrounding pixel unit. In other embodiments, the Nth closest pixel unit (e.g., N=2, 3, 4, 5, 6 . . . ) to the main pixel may be defined as the surrounding pixel unit.

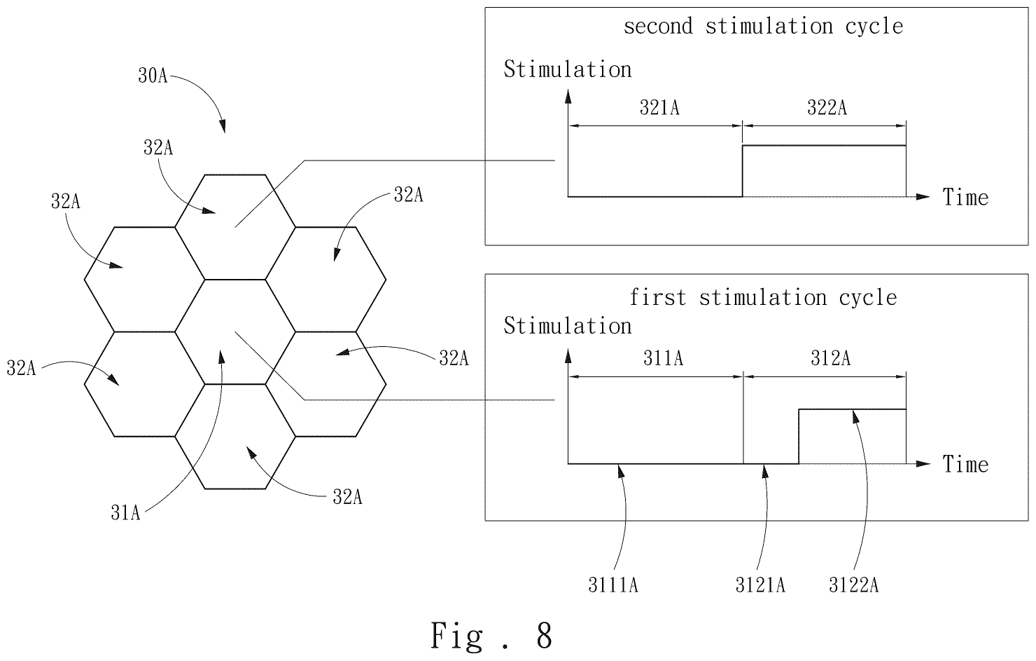

FIG. 8 illustrates a pixel group unit with stimulation cycle according to an exemplary embodiment of the invention. Taking the pixel group unit 30A as the example. The main pixel unit 31A and the surrounding pixel units 32A at different locations respectively output an electrical stimulation waveform according to different stimulation cycles to create a color perception.

The main pixel unit 31A and the surrounding pixel units 32A output a first stimulation cycle and a second stimulation cycle respectively to retinal cells of the user. The first stimulation cycle of the main pixel unit 31A has a first duration 311A and a second duration 312A after the first duration 311A. The second stimulation cycle of the surrounding pixel unit 32A has a first duration 321A and a second duration 322A after the first duration 321A.

The first duration 311A of the first stimulation cycle is [symbol] an inactive period 3111A. The second duration 312A of the first stimulation cycle has an inactive period 3121A greater than 20% and less than 80% and the rest of the second duration 312A is an active period 3122A.

The second stimulation cycle of each of the surrounding pixel units 32A also has a first duration 321A and a second duration 322A after the first duration 321A, all the first duration 321A of the second stimulation cycle is inactive period and the second duration 322A of the second stimulation cycle is entirely active period.

In the above-mentioned, the active period refers to a period that electrode of the pixel unit outputs a stimulation to the retinal cells of the user and the inactive period refers to a period that electrode of the pixel unit does not output a stimulation to the retinal cells of the user.

In the present invention, each of the pixel group units is configured to produce a specific color of vision perception at a given time. The specific color may be red, blue or green. The color of vision perception produced by the pixel group unit depends on a sequence and a length of active period and inactive period in the first-half duration of the first stimulation cycle of the main pixel unit.

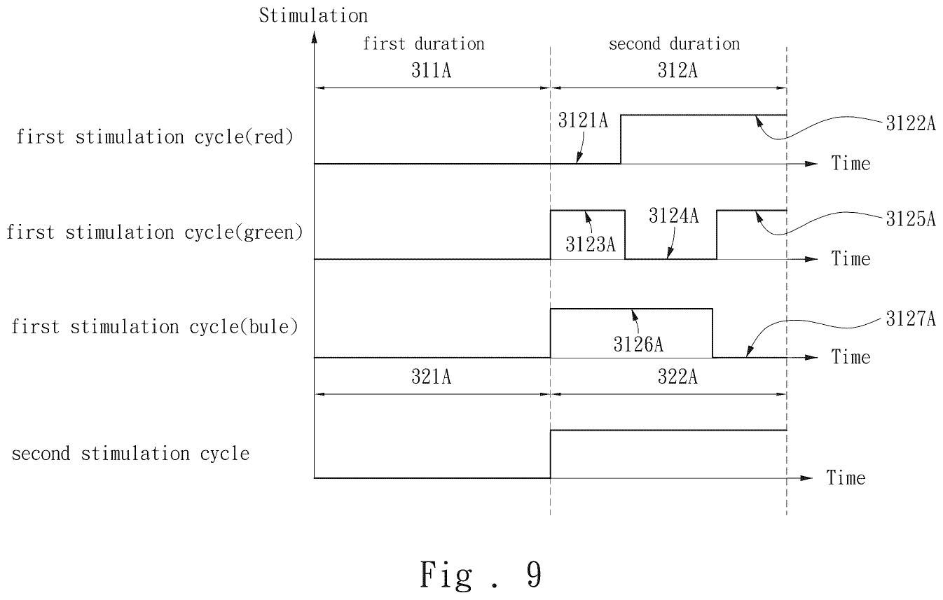

FIG. 9 illustrates various stimulation cycle for the main pixel unit and the surrounding pixel unit according to an exemplary embodiment of the invention.

In an embodiment of the invention, a red color perception can be produced when the first duration 311A of the main pixel unit 31A is entirely the inactive period, followed by the second duration 312A consists of the inactive period 3121A, which occupies approximately between 25% and 50% of the second duration 312A, and the remaining portion of the second duration 312A being the active period 3122A.

In an embodiment of the invention, a green color perception is produced when the first duration 311A of the main pixel unit 31A is entirely the inactive period, followed by the second duration 312A consists of the active period 3123A, which occupies greater than 25% of the second duration 312A, followed by the inactive period 3124A, which occupies approximately between 30% and 80% of the second duration 312A, and the remaining portion of the second duration 312A being the active period 3125A.

In an embodiment of the invention, a blue color perception is produced when the first duration 311A of the main pixel unit 31A is entirely the inactive period, followed by the second duration 312A consists of the active period 3126A, which occupies approximately between 20% and 80% of the second duration 312A, and the remaining portion of the second duration 312A being the inactive period 3127A.

It should be noted that the foregoing descriptions of stimulation cycles and the operations of the color shutter are provided as illustrative examples only, and are not intended to limit the scope of the present invention. Other definitions, variations, or implementations of stimulation cycles and color shutter operations may also be applied within the spirit of the invention. In some examples, instead of employing a (temporal) color shutter, (spatial) color filters may be used to generate color signals.

In the following, an example of the artificial retinal prosthesis will be described. The configuration of the artificial retinal prosthesis may refer to FIG. 8, reference sign 31A denotes a main pixel unit while reference signs 32A denote at least one auxiliary pixel unit adjacent to the main pixel unit 31A. The main pixel unit 31A and its auxiliary pixel units 32A form a pixel group unit 30A.

Herein, the term “its auxiliary pixel unit” may refer to the pixel unit located closest and adjacent to the main pixel unit (e.g., the nearest pixel unit 32A to the main pixel unit 31A). It should be understood that, for a given main pixel unit 31A, the “its auxiliary pixel unit” may encompass one or more auxiliary pixel units. Hereinafter, the main pixel unit 31A and its auxiliary pixel units 32A may be collectively referred to as “pixel unit.”

In the example, the pixel group unit 30A is arranged in a hexagonal tiling pattern, in which the main pixel unit 31A is surrounded by six adjacent auxiliary pixel units 32A in a honeycomb-like arrangement. However, the arrangement of the pixel group units 30A is not limited to the hexagonal tiling pattern. Other configurations, such as pixel units arranged in horizontal rows and vertical columns to form a grid-like layout, or other geometric tiling patterns, may also be employed within the scope of the invention.

In the present example, each pixel group unit 30A is defined by one main pixel unit 31A together with six adjacent auxiliary pixel units 32A arranged in the honeycomb-like manner. However, in other examples, such as the grid-like layout, a pixel group unit may alternatively be defined as including an entire horizontal row or an entire vertical column of pixel units.

In the example, the main pixel unit 31A and its auxiliary pixel units 32A are substantially the same component, i.e., they are physically identical pixel units disposed at different spatial positions. The distinction between the main pixel unit 31A and its auxiliary pixel units 32A lies in their operation, in that these pixel units are driven according to different sequences of stimulation cycles, thereby performing different functional roles within the pixel group unit 30A.

The pixel group unit 30A is configured to receive color signals that represent a visual image, such as the light depicted in FIG. 1. In other words, the color signals correspond to the light passing through the color shutter 20 (or alternatively, through color filters), which is then received by the pixel group unit 30A.

Based on the received signals, the main pixel unit 31A and its auxiliary pixel units 32A are configured to operate according to a first sequence of cycles and a second sequence of cycles respectively. As used herein, the term “sequence of cycles” refers to the temporal order of on and off periods of the pixel unit during electrical stimulation. In other words, the sequence of cycles defines the timing pattern in which the pixel unit is activated (on) and deactivated (off) to output stimulation to the retinal cells.

Accordingly, the main pixel unit 31A and its auxiliary pixel units 32A are required to be not only spatially arranged in association but also in their respective sequences of cycles. For example, the main pixel unit 31A and its auxiliary pixel units 32A may be spatially arranged in a complementary manner and coordinated in their on and off timing patterns so as to achieve the intended stimulation effect.

In the example, the main pixel unit 31A and its auxiliary pixel units 32A operate according to a first sequence of cycles and a second sequence of cycles, respectively. From another perspective, the pixel unit that executes the first sequence of cycles is defined as the main pixel unit 31A, while the pixel unit that executes the second sequence of cycles is defined as the auxiliary pixel unit 32A. In this sense, the distinction between the main pixel unit 31A and the auxiliary pixel units 32A is not based on differences in physical configuration, but rather on the respective sequence of cycles assigned to each pixel unit. Accordingly, it may also be understood that the designation of a pixel unit as the main pixel unit or the auxiliary pixel unit may be selectable. For example, a given pixel unit may serve as the main pixel unit at one time, and later as the auxiliary pixel unit, such that the roles of the main pixel units and the auxiliary pixel units can be swapped.

In other words, a first set of the pixel units may be designated as main pixel units and a second set of the pixel units may be designated as auxiliary pixel units according to whether the pixel units operate under the first sequence of cycles or the second sequence of cycles. Furthermore, the designations of the first set and the second set may be dynamically swapped between cycles.

Each of the first sequence of cycles and the second sequence of cycles comprises a dark period followed by a lighted period, such that the pixel units intermittently output lighted pulses (e.g., electrical pulse) during the lighted period, each of the dark period being of comparable duration as the lighted period. It should be understood that, in some examples, the lighted period may precede the dark period. In general, for a given pixel unit, the lighted period and the dark period may alternate with each other in time.

In one example, the term “dark period” may refer to a period in which the pixel unit does not output the lighted pulses (e.g., electrical pulse) throughout the entire duration (the dark period). However, the dark period may also refer to a period in which the pixel unit remains off for one or more predominant portions of the time, with only one or more minor portions of the time outputting the lighted pulses. For instance, the dark period may be when the pixel unit is off for more than 50% or 70% of the duration.

In one example, the term “lighted period” may refer to a period in which the pixel unit continuously outputs the lighted pulses throughout the entire duration (the lighted period). However, the lighted period may also refer to a period in which the pixel unit outputs the lighted pulses for one or more predominant portions of the time, with only one or more minor portions of the time being off. For instance, the lighted period may be when the pixel unit outputs the lighted pulses for more than 50% or 70% of the duration.

In the example, the lighted periods of the main pixel unit 31A and the lighted periods of its auxiliary pixel units 32A are synchronized, and the dark periods of the main pixel unit 31A and the dark periods of its auxiliary pixel unit 32A are synchronized. Hereinafter, the term “synchronized” may refer to the lighted periods (or the dark periods) of the main pixel unit 31A and of its auxiliary pixel units 32A having substantially the same duration, and further starting and ending (i.e., switching to the next period or cycle) at substantially the same time. It should be noted that at least one complete cycle may be sufficient to induce the color percepts. However, the number of cycles may be varied or adjusted as needed.

In one example, the artificial retinal prosthesis may be configured to provide different color percepts. As used herein, the term “color percepts” refers to chromatic or non-achromatic visual perceptions, such as reddish, greenish, bluish, yellowish, orangish, purplish, pinkish, cyan, magenta, whitish, or grayish percepts, and the like. Though the induced color percepts might be affected and thus differed among observers by the ethnics, age, gender, prior knowledge of color vision, retinal condition and sensitivity of the observer, the induced color percepts are nonetheless perceived as chromatic rather than achromatic.

In certain examples, a black color percept may also be produced when both the main pixel units 31A and the auxiliary pixel units 32A remain off.

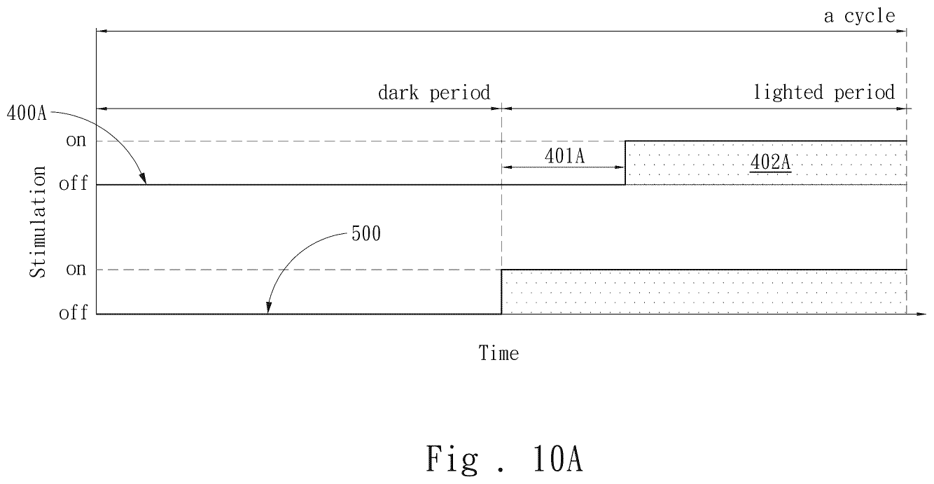

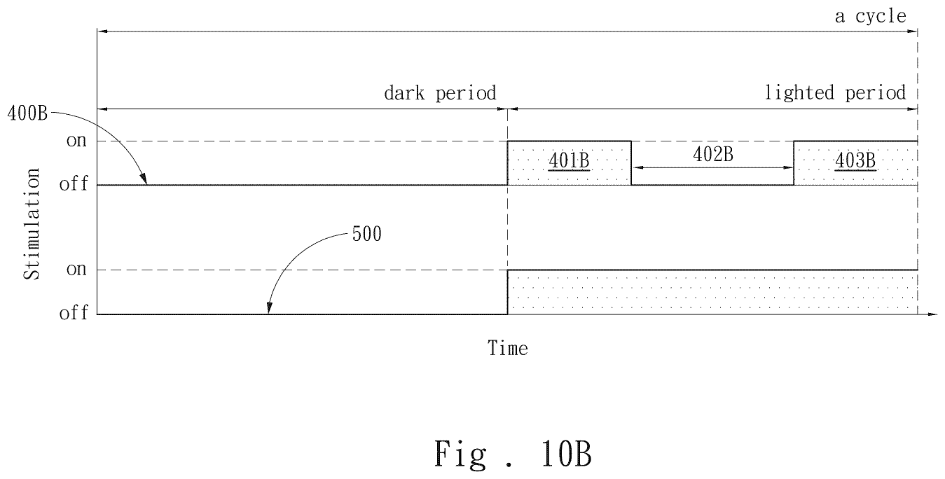

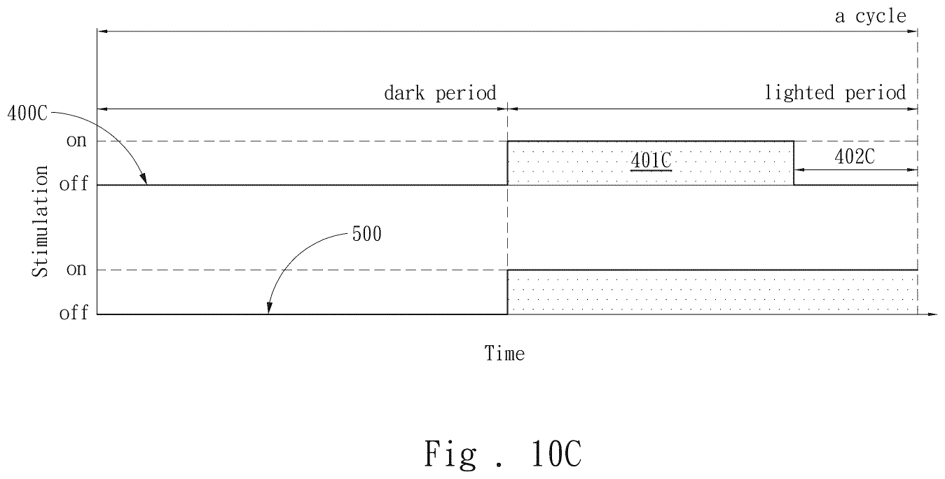

FIGS. 10A, 10B, and 10C respectively illustrate a single cycle corresponding to a reddish color percept, a greenish color percept, and a bluish color percept. In particular, reference signs 400A, 400B, and 400C respectively denote the cycles of the first sequence of cycles of the main pixel unit 31A when inducing the reddish, greenish, and bluish color percepts. Reference sign 500 denotes the cycle of the second sequence of cycles of the auxiliary pixel units 32A.

As illustrated in FIG. 10A, the reddish color percept may be induced when the main pixel units 31A are configured such that the off duration 401A is followed by the lighted duration 402A for the remaining portion of the lighted period. In one example, the off duration 401A of the lighted period lasts continuously and occupies about 25% to 50% of the total duration of the lighted period. In another example, the off duration 401A of the lighted period occupies about 25% to 37.5% of the total duration of the lighted periods.

As illustrated in FIG. 10B, the greenish color percept may be induced when the main pixel units 31A are configured such that the initial lighted duration 401B is followed by the off duration 402B, which is in turn followed by the subsequent lighted duration 403B for the remaining portion of the lighted periods. In one example, the lighted period of the main pixel unit 31A is followed by the off duration 402B that begins no earlier than about 25% and no later than about 37.5% of the total duration of the lighted period, and ends no earlier than about 62.5% and no later than about 80% of the total duration of the lighted period.

As illustrated in FIG. 10C, a bluish color percept may be induced when the main pixel units are configured such that the initial lighted duration 401C is followed by the off duration 402C for the remaining portion of the lighted periods. In one example, the lighted duration 401C of the lighted periods lasts continuously and occupies about 50% to 75% of the total duration of the lighted periods.

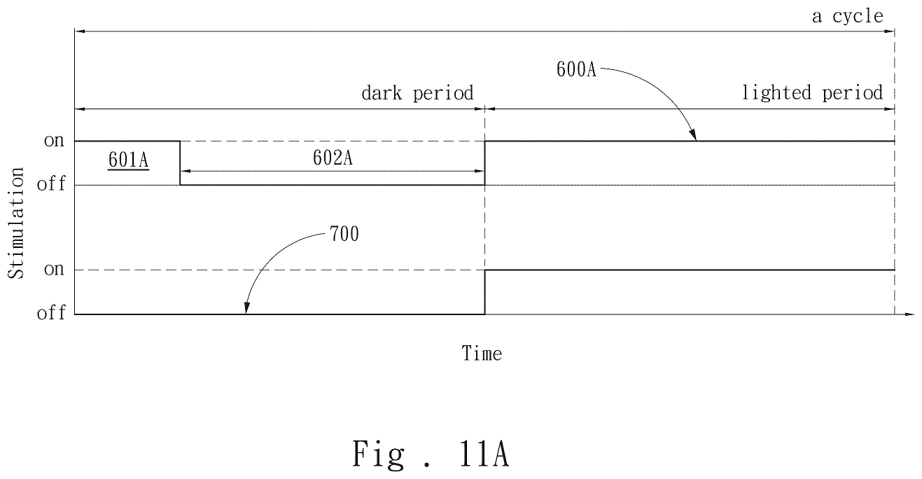

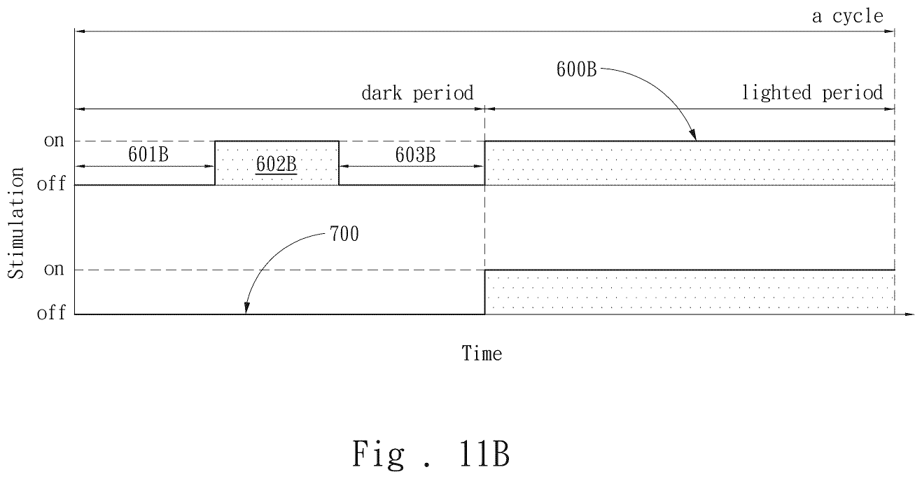

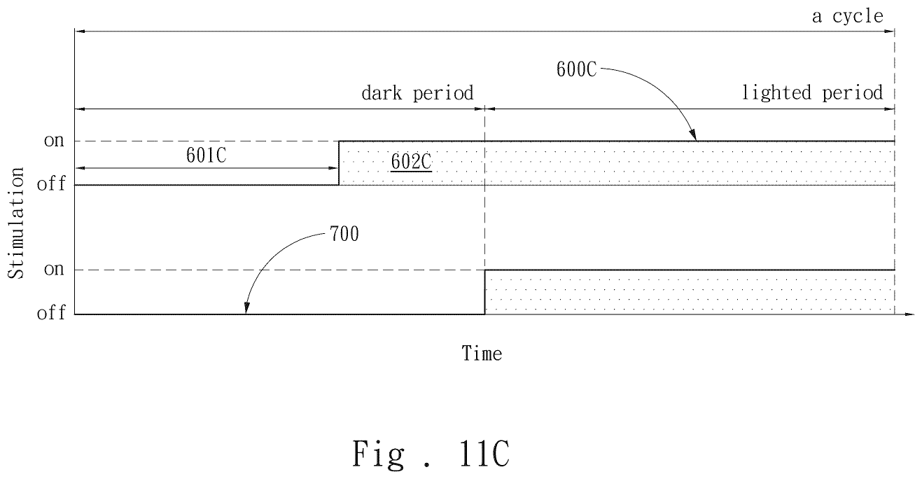

FIGS. 11A, 11B, and 11C respectively illustrate a single cycle corresponding to different stimulation patterns of the main pixel unit 31A and its auxiliary pixel units 32A. It should be noted that FIGS. 11A-11C illustrate stimulation cycles that differ from those shown in FIGS. 10A-10C. In FIGS. 10A-10C, the dark period corresponds to a state in which the pixel unit is off throughout the entire duration of the dark period, while the lighted period corresponds to a state in which the pixel unit is predominantly on with only minor off intervals. By contrast, in FIGS. 11A-11C, the lighted period corresponds to a state in which the pixel unit is on throughout the entire duration of the lighted period, whereas the dark period corresponds to a state in which the pixel unit is predominantly off with only minor on intervals.

In particular, reference signs 600A, 600B, and 600C respectively denote the lighted periods of the main pixel unit 31A in FIGS. 11A, 11B, and 11C. Reference sign 700 denotes the cycle of the second sequence of cycles of the auxiliary pixel units 32A.

As illustrated in FIG. 11A, the main pixel unit 31A is configured such that, within the dark period, the lighted duration 601A is followed by the off duration 602A, and thereafter the lighted period resumes. The auxiliary pixel units 32A are off during the dark period and are on during the lighted period. In addition, for the main pixel unit 31A, the lighted duration 601A within the dark period is shorter than the off duration 602A.

As illustrated in FIG. 11B, the main pixel unit 31A is configured such that, within the dark period, the off duration 601B is followed by the lighted duration 602B, which is in turn followed by the off duration 603B, and thereafter the lighted period resumes. The auxiliary pixel units 32A are off during the dark period and are on during the lighted period. In addition, for the main pixel unit 31A, the lighted duration 602B within the dark period is shorter than the combined off durations 601B and 603B.

As illustrated in FIG. 11C, the main pixel unit 31A is configured such that, within the dark period, the off duration 601C is followed by the lighted duration 602C, and thereafter the lighted period resumes. The auxiliary pixel units 32A are off during the dark period and are on during the lighted period. In addition, for the main pixel unit 31A, the off duration 601C within the dark period is longer than the subsequent lighted duration 602C.

In one example, the term “sequence of cycles” may refer to a repetition of multiple (same or different) cycles over time. For instance, with reference to FIG. 10A, for a given main pixel unit 31A, the sequence of cycles may comprise a repeating pattern of a dark period followed by a lighted period, such as dark period→lighted period→dark period→lighted period, and so on in succession. It should be understood that, for a given main pixel unit 31A, the sequence of cycles may correspond to a repetition of one type of cycle (e.g., the cycle shown in FIG. 10A) during a certain interval of time, but may subsequently correspond to a repetition of another type of cycle (e.g., the cycle shown in FIG. 10B).

In some examples, the dark periods shared by the main pixel units and the auxiliary pixel units may have a duration of at least about 20 milliseconds. The frequency of the dark periods may be set within a range between about 2 Hz and 24 Hz, and in other embodiments, more preferably between about 4 Hz and 20 Hz. Furthermore, the intensity of the lighted durations of the auxiliary pixel units relative to the intensity of the lighted durations of the associated main pixel units may be defined in terms of a Michelson Contrast, which may range from about 25% to 100%, and in other embodiments, more preferably from about 40% to 100%.

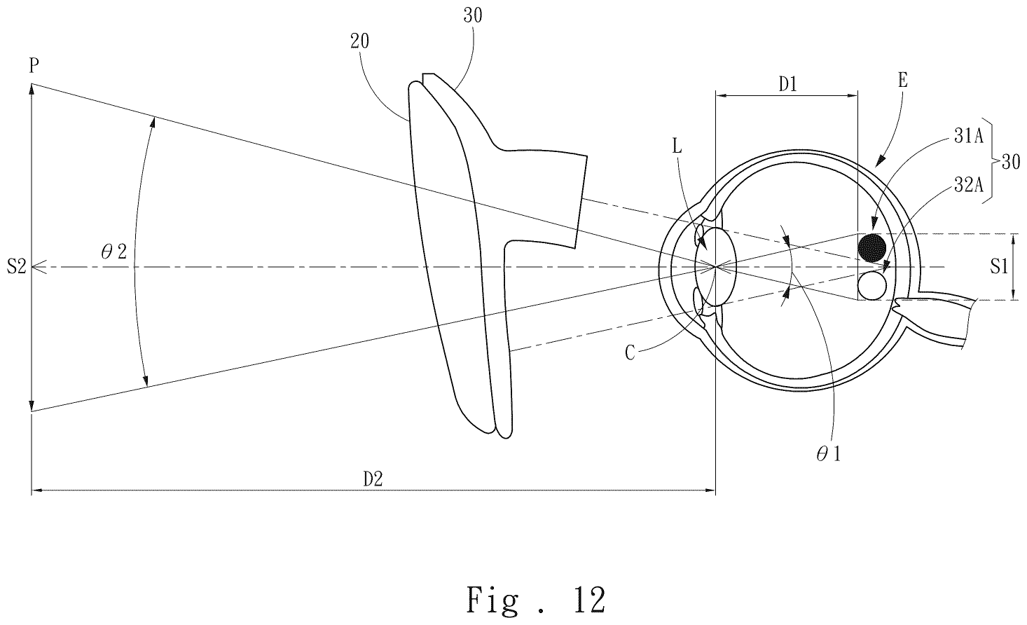

In some examples, the spacing between the pixel units may be defined in terms of a visual angle subtended by the pixel group unit. As illustrated in FIG. 12, a visual angle θ1 is defined between the main pixel unit 31A and its auxiliary pixel unit 32A of the pixel group unit as observed (or visually perceived) by a user implanted with the artificial retinal prosthesis, and reference sign E denotes the eyeball of the user. The visual angle θ1 is determined by the relationship:

$$θ 1 = {tan}^{- 1} \frac{S 1}{2 D 1} = {tan}^{- 1} \frac{S 2}{2 D 2} = θ 2 ,$$

where S1 represents the spatial extent between the main pixel unit 31A and the auxiliary pixel unit 32A, and D1 represents the distance between a center C of the crystalline lens L of the eyeball E of the user and the pixel group unit 30A of the artificial retinal prosthesis, S2 represents spatial extent between the main pixel unit 31A and the auxiliary pixel unit 32A when these pixel units were to be inversely projected toward external project plane P at a distance D2 through the crystalline lens L.

In one example, the artificial retinal prosthesis may be implanted using either an epi-retinal (e.g. from the front side of retina or on the retina) approach or a sub-retinal (e.g. behind the retina) approach.

In one example, the visual angle θ may be in a range from about 0.076° to about 2.76°. In another example, the visual angle θ may be in a range from about 0.46° to about 2.76°.

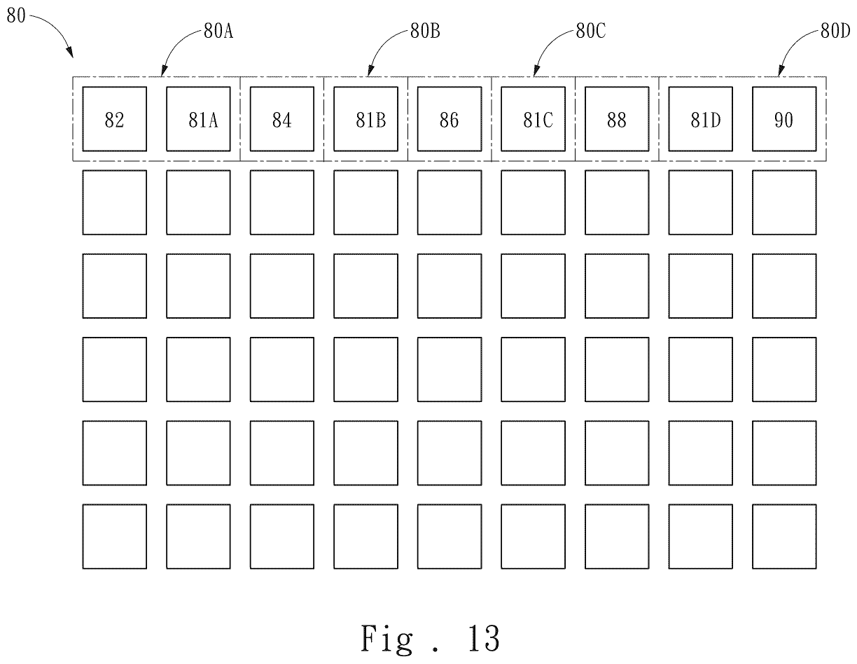

FIG. 13 illustrates an example of a configuration of the pixel group units. The pixel array 80 includes a plurality of pixel group units 80A, 80B, 80C, 80D. Each of the pixel group units includes a main pixel unit and at least one auxiliary pixel unit adjacent to the main pixel unit. In the example, reference signs 81A, 81B, 81C and 81D denote the main pixel units, while reference signs 82, 84, 86, 88 and 90 denote the auxiliary pixel units. That is, adjacent pixel group units may overlap, by way of example, the pixel group unit 80B overlaps with the pixel group units 80A and 80C. Further, the auxiliary pixel unit may be shared by two or more pixel group units. For example, the auxiliary pixel unit 84 is shared by the pixel group unit 80A and the pixel group unit 80B. In other words, reference sign 84 may be the auxiliary pixel unit of the main pixel unit 81A and also the auxiliary pixel unit of the main pixel unit 81B. Although FIG. 13 illustrates only certain auxiliary pixel units 82, 84, 86, 88, 90 for clarity, additional auxiliary pixel units may also be included adjacent to each main pixel unit.

What is claimed is:

1. An artificial retinal prosthesis to electrically stimulate a retina of an eye by a spatiotemporal electrical stimulation, comprising:
a plurality of pixel group units arranged in an array, each of the pixel group units comprising a main pixel unit and at least one auxiliary pixel unit adjacent to the main pixel unit, each of the pixel units having one or more pixels, wherein the main pixel unit and its auxiliary pixel unit receive signals that represent a visual image;
wherein the main pixel unit and its auxiliary pixel unit are configured to operate according to a first sequence of cycles and a second sequence of cycles respectively, each cycle comprising a dark period followed by a lighted period, such that the pixel units intermittently output lighted pulses during the lighted period, each of the dark period being of comparable duration as the lighted period;
wherein the lighted periods of the main pixel unit and the lighted periods of its auxiliary pixel unit are synchronized, and the dark periods of the main pixel unit and the dark periods of its auxiliary pixel unit are synchronized;
wherein during the lighted periods, the main pixel unit is nominally turned on for at least one lighted duration except for an off duration while the auxiliary pixel units are continuously turned on for the entire lighted period, and wherein during the dark periods, both the main pixel unit and the auxiliary pixel units are continuously turned off for the entire dark period; and
wherein the artificial retinal prosthesis is configured to provide at least one of the following color percepts:
reddish color when the main pixel units are configured such that the off duration of the lighted periods followed by the lighted duration for the remaining of the lighted periods of its main pixel units;
greenish color when the main pixel units are configured such that the initial lighted duration is followed by the off duration, which is in turn followed by the subsequent lighted duration for the remaining portion of the lighted periods; or
bluish color perceived when the main pixel units are configured such that the lighted duration of the lighted periods followed by the off duration for the remaining of the lighted periods of its main pixel units;
wherein at least one complete cycle is required to induce the color percepts.

2. The artificial retinal prosthesis as claimed in claim 1, wherein the reddish color is perceived when the off duration of the lighted periods lasts continuously and occupies 25% to 50% of the total duration of the lighted periods.

3. The artificial retinal prosthesis as claimed in claim 1, wherein the reddish color is perceived when the off duration of the lighted periods occupies 25% to 37.5% of the total duration of the lighted periods.

4. The artificial retinal prosthesis of claim 1, wherein the greenish color is perceived when the lighted duration of the lighted period of the main pixel unit is followed by an off duration that falls no earlier than 25% and no later than 37.5% of the lighted period of the main pixel unit, and rises no earlier than 62.5% and no later than 80% of the lighted period of the main pixel unit.

5. The artificial retinal prosthesis of claim 1, wherein the bluish color is perceived when the lighted duration of the lighted periods lasts continuously and occupies 50% to 75% of the total duration of the lighted periods.

6. The artificial retinal prosthesis of claim 1, wherein black color is perceived when the main pixel units and the auxiliary units are both off.

7. The artificial retinal prosthesis of claim 1, wherein the dark periods shared by the main pixel units and the auxiliary pixel units have a duration of at least 20 milliseconds.

8. The artificial retinal prosthesis of claim 1, wherein a visual angle (θ) subtended by at least one of the pixel group units satisfies
$$θ = {tan}^{- 1} \frac{S}{2 D} ,$$
where S represents a spatial extent between the main pixel unit and its auxiliary pixel unit of the pixel group unit, and D represents a distance between a center of the crystalline lens of the eyeball of the user implanted with the artificial retinal prosthesis, and wherein θ is in a range from 0.076° to 2.76°.

9. The artificial retinal prosthesis as claimed in claim 8, wherein θ is in a range from 0.46° to 2.76°.

10. The artificial retinal prosthesis as claimed in claim 1, wherein the plurality of pixel group units are formed by a plurality of pixel units arranged in the array, each of the pixel units being substantially structurally identical, wherein a first set of the pixel units is designated as main pixel units and a second set of the pixel units is designated as auxiliary pixel units according to whether the pixel units operate under the first sequence of cycles or the second sequence of cycles, and wherein the designations of the first set and the second set are configured to be swapped between cycles.

11. The artificial retina prosthesis as claimed in claim 1, wherein the frequency of the dark periods is between 2 Hz and 24 Hz.

12. The artificial retina prosthesis as claimed in claim 1, wherein the frequency of the dark periods is between 4 Hz and 20 Hz.

13. The artificial retina prosthesis of claim 1, wherein an intensity of the lighted durations of the auxiliary pixel units and an intensity of the lighted durations of the associated main pixel units have a Michelson Contrast in a range from 25% to 100%.

14. The artificial retina prosthesis of claim 1, wherein an intensity of the lighted durations of the auxiliary pixel units and an intensity of the lighted durations of the associated main pixel units have a Michelson Contrast in a range from 40% to 100%.

15. The artificial retina prosthesis of claim 1, wherein the main pixel units and the auxiliary pixel units are grouped in respective rows, with the rows of main pixel units being spaced apart by the rows of auxiliary pixel units.

16. The artificial retina prosthesis of claim 1, wherein different sets of the pixel group units are configured to provide different color percepts simultaneously.

17. An artificial retinal prosthesis to electrically stimulate a retina of an eye by a spatiotemporal electrical stimulation, comprising:
a plurality of pixel group units arranged in an array, each of the pixel group units comprising a main pixel unit and at least one auxiliary pixel unit adjacent to the main pixel unit, each of the pixel units having one or more pixels, wherein the main pixel unit and its auxiliary pixel unit receive signals that represent a visual image;
wherein the main pixel unit and its auxiliary pixel unit are configured to operate according to a first sequence of cycles and a second sequence of cycles respectively, each cycle comprising a lighted period followed by a dark period, such that the pixel units intermittently output lighted pulses during the lighted period, each of the dark period being of comparable duration as the lighted period;
wherein the lighted periods of the main pixel unit and the lighted periods of its auxiliary pixel unit are synchronized, and the dark periods of the main pixel unit and the dark periods of its auxiliary pixel unit are synchronized;
wherein during the lighted periods, the main pixel unit and the auxiliary pixel units are continuously turned on for the entire lighted period, and during the dark periods, the main pixel unit is nominally turned off for at least one off duration except for a lighted duration while the auxiliary pixel units is continuously turned off for the entire dark period; and
wherein the artificial retinal prosthesis is configured to provide at least one of the following color percepts:
first color when the main pixel units are configured such that the off duration of the lighted periods followed by the lighted duration for the remaining of the lighted periods of its main pixel units;
second color when the main pixel units are configured such that the initial lighted duration is followed by the off duration, which is in turn followed by the subsequent lighted duration for the remaining portion of the lighted periods; or
third color when the main pixel units are configured such that the lighted duration of the lighted periods followed by the off duration for the remaining of the lighted periods of its main pixel units;
wherein at least one complete cycle is required to induce the color percepts.

18. The artificial retina prosthesis of claim 17, wherein black color is perceived when the main pixel units and the auxiliary units are both off.

19. The artificial retina prosthesis of claim 17, wherein different sets of the pixel group units are configured to provide different color percepts simultaneously.
